(19) United States
(12) Patent Application Publication
(10) Pub. No.: US 2021/0128082 A1
(43) Pub. Date: May 6, 2021

(54) METHODS AND SYSTEMS FOR BODY CONTOUR PROFILES

(71) Applicant: GE Precision Healthcare LLC, Milwaukee, WI (US)

(72) Inventors: Talia Cohen, Nesher (IL); Ken Efrati, Kiriat Motzkin (IL)

(21) Appl. No.: 16/676,285

(22) Filed: Nov. 6, 2019

(51) Int. Cl. A61B 6/03; A61B 6/04; A61B 6/00; G01R 33/54

(52) U.S. Cl. CPC A61B 6/037; A61B 6/0407; A61B 6/5294; G01R 33/543; A61B 6/527; A61B 6/032; A61B 6/545; A61B 6/5247

(57) ABSTRACT

Methods and systems are provided for generation and storage of body contour profiles during an imaging procedure. In one embodiment, an imaging system may include a plurality of contour sensors installed along one or more rings mounted on a gantry of the imaging device along a perimeter of a bore of the gantry. A body contour profile of a subject may be generated and saved prior to and during the scan. A visualization of the body contour profile may be used by an operator of the imaging system to adjust scan parameters such as detector positions during the scan. In this way, a real-time visualization of a body contour of a subject may be used by the operator to control the imaging procedure and generate improved medical images.

### FIELD

Embodiments of the subject matter disclosed herein relate to non-invasive diagnostic imaging, and more particularly, to generation and storage of body contour profiles during an imaging procedure.

### BACKGROUND

Medical imaging systems are used to assist with diagnosis of medical ailments in patients by generating image data showing internal elements of the patients, such as organs, bones, blood, and the like. Some medical imaging systems such as nuclear medicine (NM) imaging system involves the application of radioactive substances (such as tracers) into the patient. Single Photon Emission Computed Tomography (SPECT) and Positron Emission Tomography (PET) are examples of nuclear imaging systems. A goal of nuclear medicine imaging systems is to provide high quality images of a patient to physician for analysis, while limiting the radiation exposure of an operator (e.g., an imaging technician) that administers the imaging scan.

In order to achieve high quality images of a patient is to position detection heads of the NM imaging system close to the patient during an imaging scan or procedure. Patient position with reference to the imaging system may be adjusted prior to and during the scan by the operator. Also, relative positioning of the detector heads with the patient is to be setup and monitored by the operator.

### BRIEF DESCRIPTION

In one embodiment, a method for an imaging device, comprises: prior to a start of an imaging scan, retrieving an initial body contour profile of a subject to be scanned, setting up scan parameters for the imaging scan based on the retrieved body contour profile, generating body contour profile during the imaging scan, providing visualization of the generated body contour profile to an operator, and the operator adjusting the scan parameters based on the generated body contour profile. In this way, by saving the geometric body contour profile of each patient, patient positioning prior to and during the scan may be improved.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 is a pictorial view of an exemplary imaging system according to an embodiment of the invention.

FIG. 2 is a perspective view of an imaging apparatus of the imaging system, according to an embodiment of the invention.

FIG. 3 is a perspective view of a portion of the imaging apparatus shown in FIG. 2 with detector arms in a retracted position relative to a gantry.

FIG. 4 shows a block diagram of the imaging system.

FIG. 5 shows the imaging apparatus of the imaging system during a contour scan.

FIG. 6 shows a gantry including multiple rings of contour sensors.

FIG. 7A is a pictorial view of an exemplary imaging system including two rings of contour sensors.

FIG. 7B is a pictorial view of sagging of a bed within a gantry of the imaging system.

FIG. 8 shows a display device of the imaging system and an enlarged inset view showing a portion of a display screen of the display device displaying a gantry visualization.

FIG. 9 is a diagram showing a side view of a human patient that represents a subject lying on a bed.

FIG. 10A shows a lateral view of an example body contour of a patient.

FIG. 10B shows a frontal view of the example body contour of the patient.

FIG. 11 is a high-level flowchart illustrating an example method for use of an example body contour of a patient during an imaging procedure.

### DETAILED DESCRIPTION

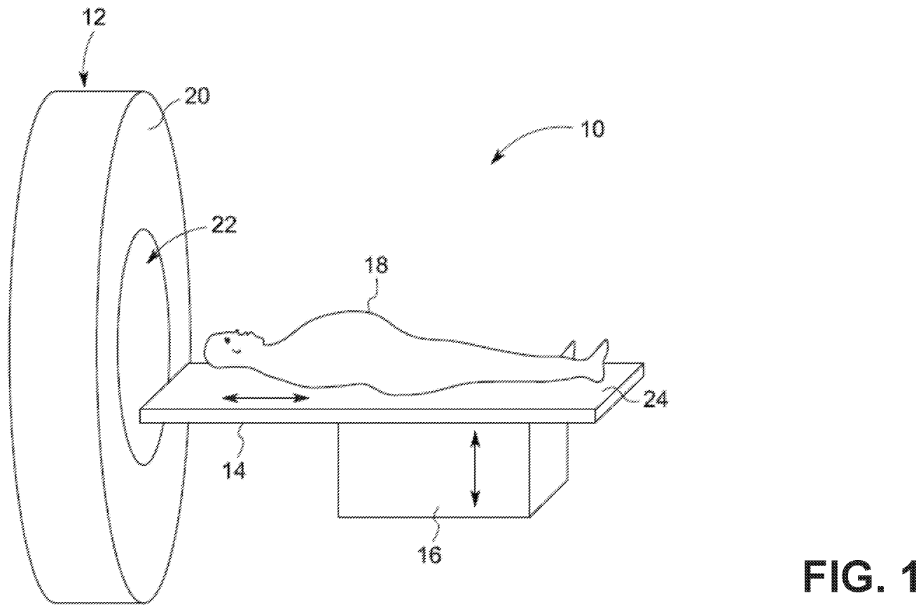

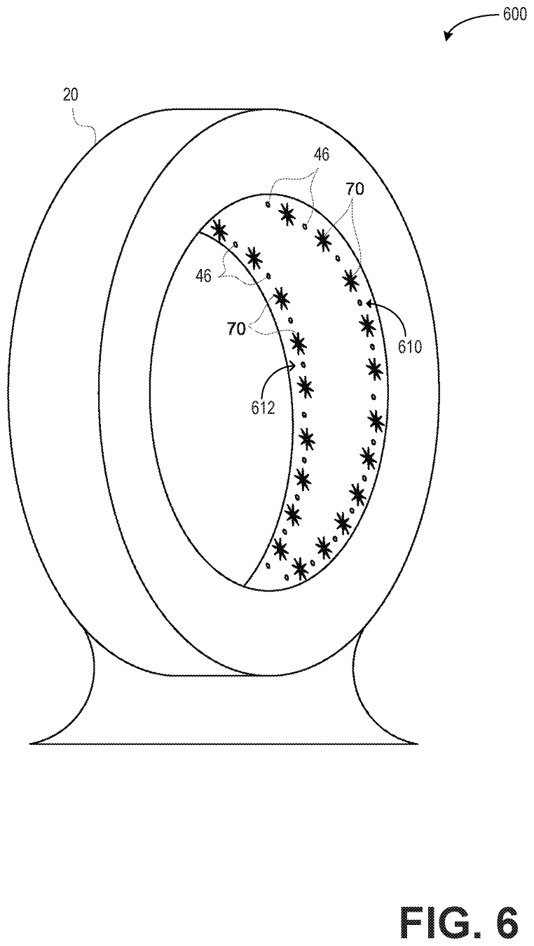

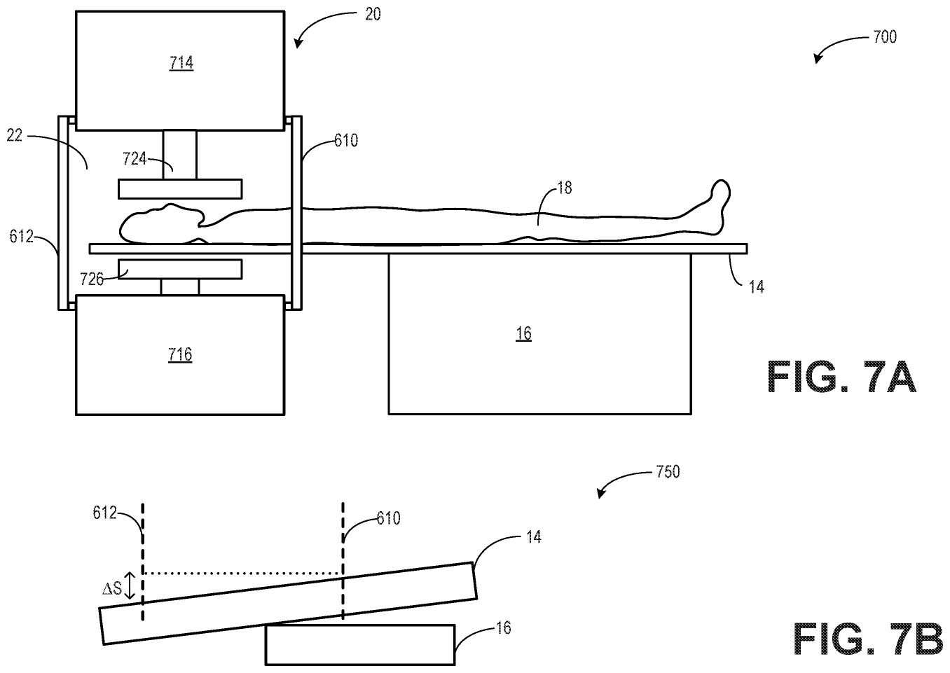

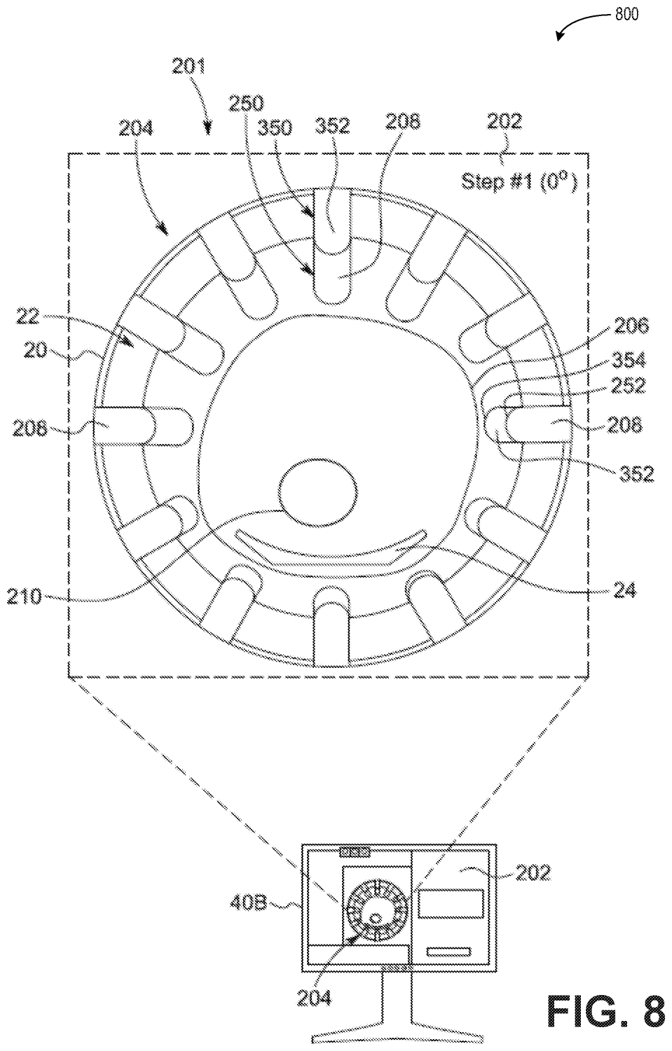

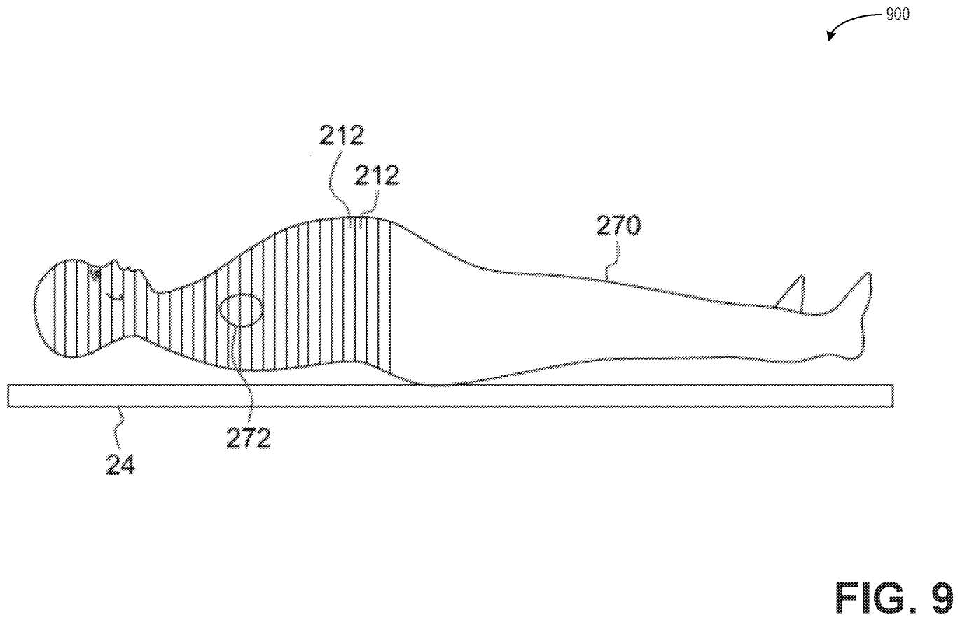

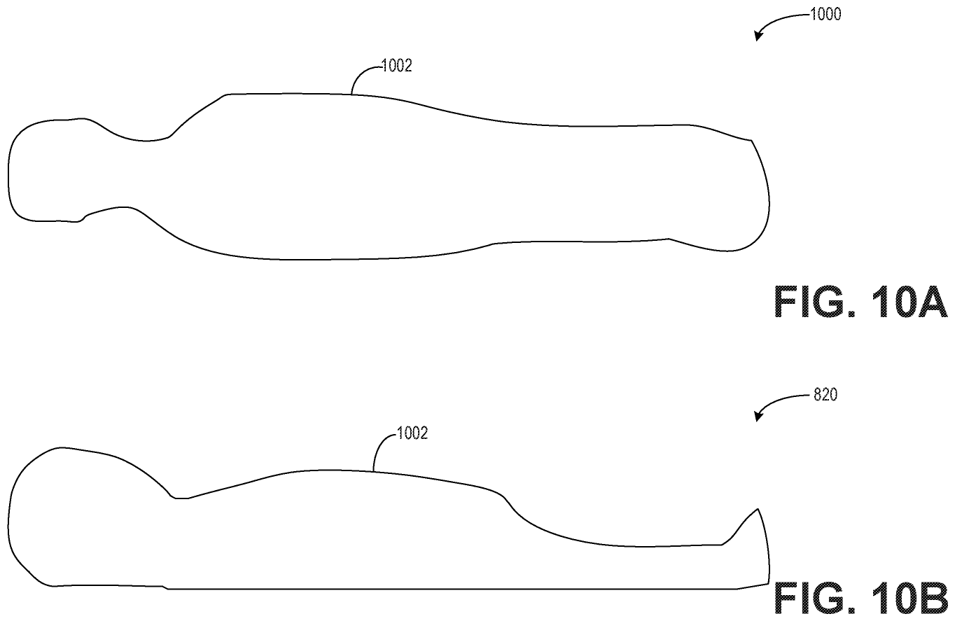

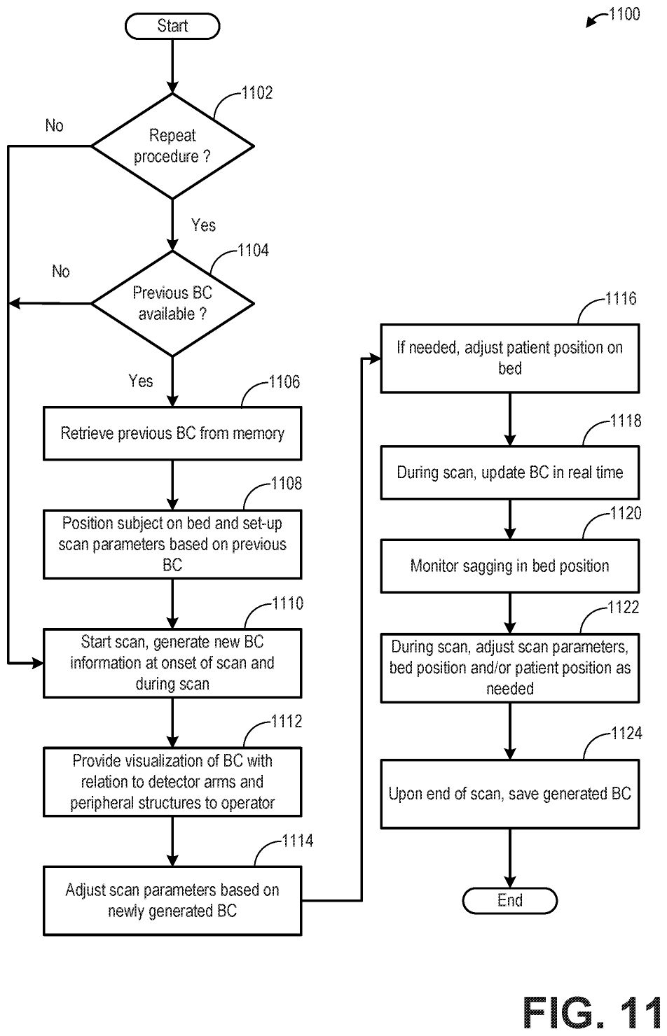

The following description relates to various embodiments of medical imaging systems. In particular, methods and systems are provided for non-invasive diagnostic imaging, and more particularly, to generation and storage of body contour profiles during a nuclear medicine (NM) imaging procedure such as Single Photon Emission Computed Tomography (SPECT) and Positron Emission Tomography (PET). An example of a NM imaging system including detector arms and contour sensors are shown in FIGS. 1-5. The contour sensors may be positioned on the imaging system in one or more rings around the bore of a gantry of the imaging device as shown in FIGS. 6 and 7A. A sagging of a bed during a scan of a subject within the gantry is pictorially depicted in FIG. 7B. A display device showing a visualization of the gantry and a body contour, as visible to an operator (such as a technician) via a display device, is shown in FIG. 8. Example views of a generated and saved body contour of a patient is shown in FIGS. 9-10A, B. FIG. 11 shows an example method for generating, saving, and using a body contour of a patient during an imaging procedure.

Though a NM imaging system is described by way of example, it should be understood that the present techniques may also be useful when applied to images acquired using other imaging modalities, such as CT, tomosynthesis, MRI, C-arm angiography, and so forth. The present discussion of a NM imaging modality is provided merely as an example of one suitable imaging modality.

As used herein, the phrase “reconstructing an image” is not intended to exclude embodiments of the present invention in which data representing an image is generated but a viewable image is not. Therefore, as used herein the term “image” broadly refers to both viewable images and data representing a viewable image. However, many embodiments generate, or are configured to generate, at least one viewable image.

In one example, for a multi arm detector, a radial position of the detector is to be determined and a sweep plan for the detector arms may be formulated to optimize the patient scan without undesired scan of areas outside the patent perimeter. Also, in proximity methods, it is desired to adjust a height of a platform (also referred herein as table/bed) on which the patient is positioned during the scan to align the body contour of the patient and the detector arms. The scan motion of the detector arms may be adjusted, via inputs from the operator, based on a body contour of the patient. The operator may manually setup scan parameters based on characteristics of the patient (such as patient height, orientation) and presence of extenders or components aligned along the scan apparatus. Human setup may cause errors which may adversely affect the scan quality. Also, during the scan, due to the radiation exposure in the scanning room, the operator may remain outside the room and if there is change in position of the patient or sagging of the platform, the operator may not be aware of such changes. A change in position that is not being rectified may result in a collision between a detector arm and the patient and cause induration in the scan. Therefore, it is desirable to generate a body contour of the patient in real time during the scan and use the body contour to determine patient position relative to the detector arms.

A body contour may be generated during a scan with multi detector arms of a NM imaging system via contour sensors. The operator can view the visualization of the body contour on a display device remote from the imaging system, which enables the operator to monitor a medical imaging procedure while being out of the room housing the imaging system to avoid radiation exposure.

Prior to the scan, the operator may setup the radial positions of the detector arms based on the patient position (or series of positions during the scan), the bed position (or series of positions during the scan), and patient preference (such as proximity to a detector arm). Based on the available body contour information, the operator may set up a detector sweep plan such that the imaging is carried out within the perimeter of the patient (without wastage of energy and time by scanning outside the patient perimeter).

In addition, the medical imaging system may update the visualization of the body contour profile in real-time to show any detected changes in the subject shape outline and/or detector arm positioning. The medical imaging system described herein allows the operator to track the progress and activity of the medical imaging procedure from a separate room, while retaining an ability to intervene in the medical imaging procedure via the use of a user input device to make adjustments, communicate with the subject, or the like. For example, if the subject deviates from an original position during the image acquisition stage, the medical imaging system may notify the operator, and the operator may reposition the subject (by physically going to the imaging room or by sending instructions to the subject via an audio system) or may reposition the detector arms to achieve a desired proximity between the detector arms and the subject to enable high quality image generation.

By viewing the body contour in lateral view, the operator may identify patient position (such as positioning of head, feet, etc.) set up scan parameters based on relative positioning of the patient organs. Also, the operator may view positioning of accessories (such as head rest, leg support, other medical devices attached to the patient, etc.) and adjust radial position of the detector arms accordingly.

The body contour profile may be collected by two sets of contour sensors coupled to two contour rings. While one contour ring may be concentric with the gantry of the imaging system, a second contour ring may be located around the bed where the bed overlaps with a support. Sagging of the bed, when the patient enters the gantry (without the underlying support) may be determined based on comparisons between the body contour captured by the first ring and the body contour captured by the second ring. By effectively detecting sagging in a bed, appropriate compensating measures may be undertaken such as by adjusting the sweep plan for the detector arms.

Each time, a body contour is generated for a patient, the body contour profile may be saved along with relative positioning of the patient on the bed and the scan parameters (such as the sweep plan). If the patient returns for a follow-up scan, the saved body contour may be retrieved from memory and the patient may be positioned on the bed and relative to the detector arms as the prior scan such that the scan can be duplicated and the results may be better compared.

FIG. 1 shows a medical imaging system 10 including a medical imaging apparatus 12 and a bed 14. The medical imaging apparatus 12 includes a gantry 20 that defines a bore 22. The bed 14 includes a platform 24 that supports a subject 18 (such as a patient to be scanned), which is a human patient in the illustrated embodiment but is not limited to a human patient. For example, the subject 18 may be another living animal or an inanimate object in an alternative embodiment. The bed 14 is able to move the platform 24 longitudinally into the bore 22 for locating the subject 18 in a designated imaging position. The bed 14 includes a lift mechanism 16 that vertically raises and lowers a platform 24 for vertically positioning the subject 18 relative to the gantry 20. The lift mechanism 16 also provides a support for the platform 14 when the subject is outside the gantry 20. The medical imaging apparatus 12 includes imaging components and devices mounted to the gantry 20 for generating medical image data of the subject 18, such as image data depicting internal elements of the subject 18.

A first ring including contour sensors may also be coupled to the gantry to capture body contour information of the subject 18 within the gantry and during the scan. A second ring (not shown) including contour sensors may be positioned around the lift mechanism 16 to capture body contour information of the subject 18 prior to entering the gantry 20 and before initiation of a scan.

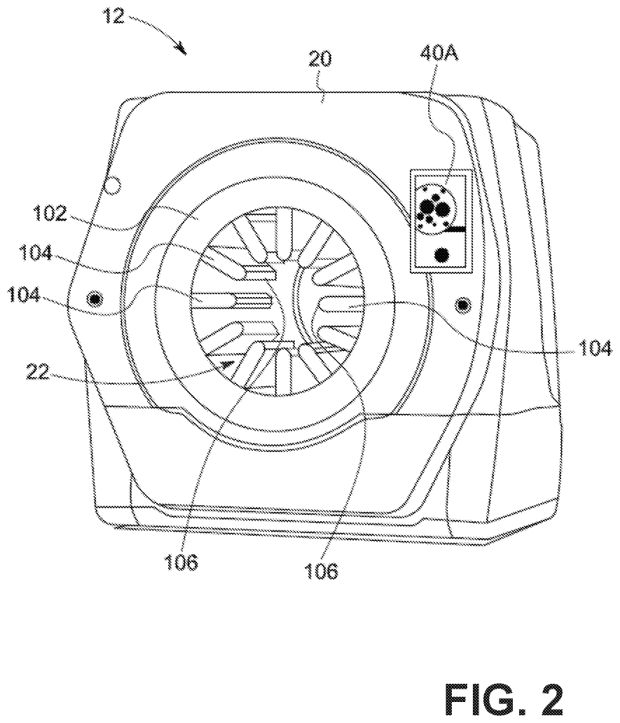

FIG. 2 is a perspective view of the medical imaging apparatus 12 of the medical imaging system 10 (shown in FIG. 1) according to an embodiment. The medical imaging apparatus 12 includes the gantry 20 which defines the bore 22. The bore 22 is open along a front side 102 of the gantry 20, and the subject on the bed 14 is received into the bore 22 across the front side 102. As used herein, relative or spatial terms such as “front,” “rear,” “top,” “bottom,” “upper,” and “lower” are used to identify and distinguish the referenced elements according to the illustrated orientations and do not necessarily require particular positions or orientations relative to the surrounding environment of the medical imaging system 10. The bore 22 has a circular cross-sectional perimeter shape in the illustrated embodiment, but may have a different shape such as elliptical or oval in an alternative embodiment.

The gantry 20 includes a plurality of detector arms 104 that are mounted along the perimeter of the bore 22. The detector arms 104 perform the medical imaging scan or procedure by detecting particles and/or radiation used to generate image data. The detector arms 104 are circumferentially spaced apart from one another along the perimeter of the bore 22. Optionally, the detector arms 104 may be uniformly arranged or distributed along the perimeter, such that the spacing between adjacent detector arms 104 is constant around the entire perimeter. The gantry 20 in the illustrated embodiment includes twelve detector arms 104, but the gantry 20 may have more or less than twelve in an alternative embodiment. In a few non-limiting examples of alternative embodiments, the gantry 20 may have three, four, five, six, eight, ten, or fourteen detector arms 104 circumferentially spaced along the perimeter of the bore 22.

In the illustrated embodiment, the medical imaging apparatus 12 is a nuclear medicine (NM) imaging apparatus, such as a SPECT system or a PET system. For example, the detector arms 104 are NM cameras configured to detect and measure radiation emitted from the subject while the subject is located at least partially within the bore 22. For example, a radioactive tracer may be administered to the subject such that the tracer is within an internal element of the subject, such as bone, organ, blood, or the like. In SPECT systems, the detector arms 104 detect and measure gamma rays that are emitted by the radioactive tracer. The detected gamma rays are used by the SPECT system to construct image data depicting the internal element of the subject. In PET systems, the radioactive tracer decays to produce positrons, and the detector arms 104 monitor photons resulting from collisions between the positrons and electrons within the subject. The medical imaging apparatus 12 may be a SPECT system, a PET system, or another type of NM system.

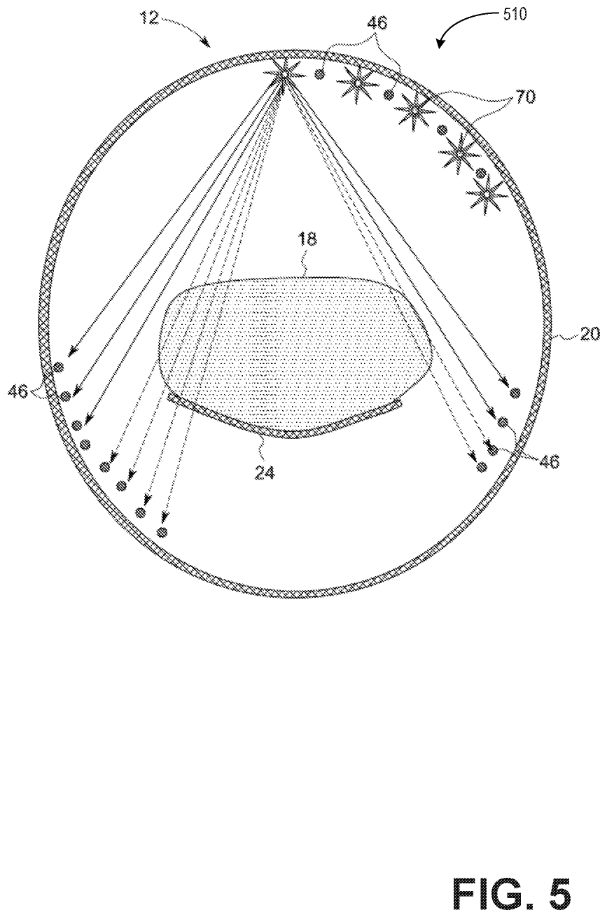

The detector arms 104 are radially movable (e.g., extendable) relative to the gantry 20, such that the detector arms 104 can be controlled to radially towards a center of the bore 22 and to move away from the center of the bore 22. For example, during a medical imaging scan, the detector arms 104 may be controlled to move towards and/or away from the subject disposed within the bore 22. In the illustrated embodiment, the detector arms 104 are shown in an extended position relative to the gantry 20. In the extended position, the detector arms 104 project from the gantry 20 into the bore 22. The detector arms 104 are radially movable to position the detector arms 104 proximate to the subject. In NM imaging, the image resolution and quality diminish with increasing distance of the detectors from the target region of interest within the subject, such as the heart in an example. The detector arms 104 are movable to allow the detector arms 104 to be retracted away from the subject while the subject is being loaded and unloaded relative to the bore 22, and to extend towards the subject during the imaging scan to be proximate to the subject for generating high resolution and high quality images of internal element(s) of the subject. Prior to a scan, the operator may set up a detector sweep plan based on the anatomy of the subject to be scanned, a position of the subject on the bed and relative to the bore 22. A body contour profile of the subject may be generated based on inputs from contour sensors coupled to the gantry and the body contour profile may be used by the operator to determine an optimal sweep plan. FIG. 5 shows an example acquisition of data by the contour sensors for generating a body contour as shown in FIGS. 9-10A, B.

The detector arms 104 may include detection heads at or proximate to distal ends 106 of the detector arms 104. The detection heads represent the one or more devices used for monitoring, detecting, capturing, measuring, filtering, and guiding the particles and/or radiation (e.g., gamma rays) emitted from the subject that are processed for generating the medical image data. The detection heads may include single crystal detectors, multi-crystal detectors, pixelated detectors, and/or scintillator-based detectors that are configured to acquire NM image data, such as SPECT image data, based on radiation emitted from the subject. The detection heads may include various semiconductor materials or non-semiconductor crystal scintillator materials. The detection heads may include collimators. The detection heads may be housed within the detector arms 104 at or proximate to the distal ends 106.

The gantry 20 optionally includes a display device 40 mounted along the front side 102. The display device 40 is viewable to an operator administering the medical imaging procedure for the subject. As used herein, the operator is broadly intended to include human persons that utilize and interact with the medical imaging apparatus 12 within the scope of employment, such as technicians, technologists, doctors, nurses, technology maintenance workers, and the like.

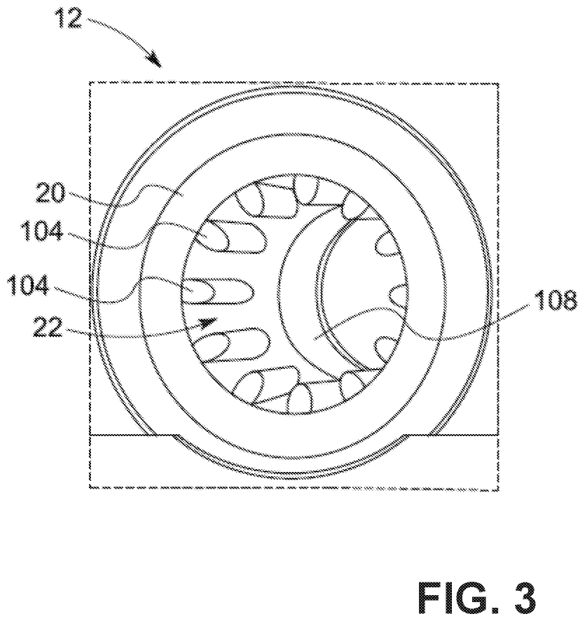

FIG. 3 is a perspective view of a portion of the medical imaging apparatus 12 shown in FIG. 2 showing the detector arms 104 in a retracted position relative to the gantry 20. Compared to the extended position shown in FIG. 2, the detector arms 104 extend a shorter distance or length into the bore 22 when in the retracted position. The detector arms 104 may be independently movable such that different detector arms 104 may extend different lengths or distances into the bore 22 in order to extend close to the subject, as it is recognized that the shape or contour of the subject is typically irregular (e.g., not perfectly cylindrical).

The medical imaging apparatus 12 may optionally include an additional imaging modality device 108 as an add-on. For example, the additional imaging modality device 108 may be a computed tomography (CT) camera which is mounted to the gantry 20 rearward of the detector arms 104 along an axial length of the bore 22.

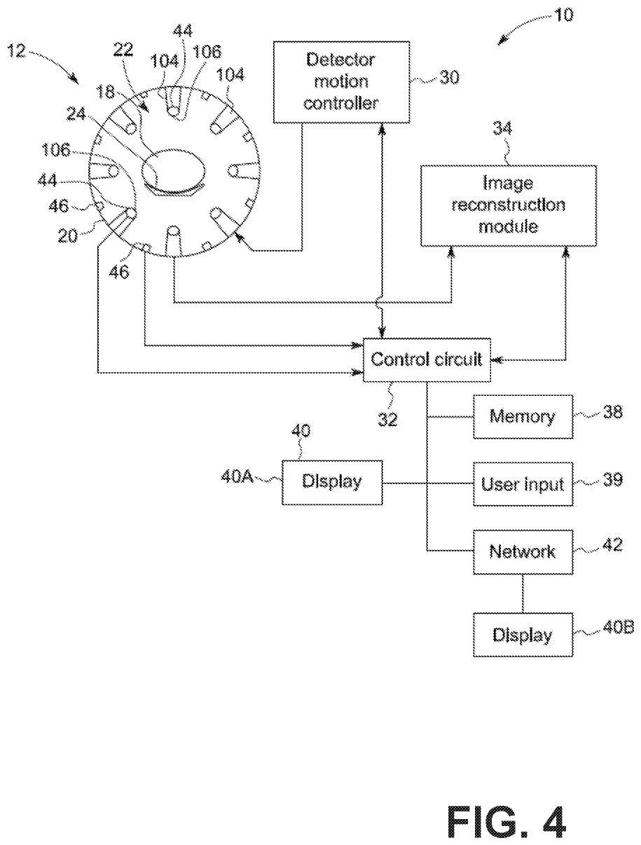

FIG. 4 is a block diagram of the medical imaging system 10 according to an embodiment. FIG. 4 also illustrates a front end view of the gantry 20, showing a subject 18 on the platform 24 within the bore 22. The gantry 20 in the illustrated embodiment has eight detector arms 104 circumferentially spaced along the perimeter of the bore 22, but alternatively may have twelve detector arms 104 as shown in FIGS. 2 and 3. The medical imaging system 10 includes the gantry 20, at least one display device 40, and a control circuit 32.

The control circuit 32 (also referred herein as controller) includes one or more processors and associated circuitry. For example, the control circuit 32 includes and/or represents one or more hardware circuits or circuitry that include, are connected with, or that both include and are connected with one or more processors, controllers, and/or other hardware logic based devices. The control circuit 32 may include a central processing unit (CPU), one or more microprocessors, a graphics processing unit (GPU), or any other electronic component capable of processing inputted data according to specific logical instructions. The control circuit 32 may be operably connected to a memory storage device 38 (referred to herein as memory 38). The memory 38 is a tangible and non-transitory computer readable medium. The memory 38 may include or represent a flash memory, RAM, ROM, EEPROM, and/or the like. The control circuit 32 may execute programmed instructions stored on the memory 38 or stored on another tangible and non-transitory computer readable medium. For example, the control circuit 32 may be configured to generate a gantry visualization that shows a subject-gantry relationship for display on one or more of the display devices 40 by executing the programmed instructions stored on the memory 38. The memory 38 optionally may store additional information that is accessible to and utilized by the control circuit 32 as described herein, such as databases, look-up tables, mathematical equations, calibration constants, and/or the like.

For example, the memory 38 may store a patient chart specific to the subject 18 and/or a patient database that contains information aggregated from historical data on patients other than the subject 18. In one example, a body contour information of a patient including position of the patient on the bed during an imaging scan may be stored in the memory. For a repeat scan of the same patient and the same anatomy (such as stress scans in cardiology, perfusion for lung scan, etc.), the body contour information may be retrieved and the patient may be positioned in the same way as before on the bed to reproduce the previous scan. Also, sweep plans and other scan parameters from a previous scan of the same anatomy of the same patient, stored in the memory, may be retrieved and used during a scan such that results from successive scans may be better compared. At a follow-up visit of a patient, the controller 32 may determine the scan range of the current scan based on the previously stored scan range and body contour information. As such, even if the patient is not positioned in the exact same way on the bed, the distance between the head and the detectors may be adjusted based on previously stored scan range and body contour information to reproduce the previous scan as closely as possible. Also, by using saved body contour information and scan parameters, the operator may reduce the time needed for setting up a scan.

Optionally, the control circuit 32 and the memory 38 may be integrated components of the medical imaging apparatus 12. For example, the control circuit 32 and memory 38 may be parts of an onboard computing device mechanically housed in or on the gantry 20. The onboard computing device may include the display 40A (also shown in FIG. 2). The control circuit 32 is communicatively connected to the at least one display device 40 via wired or wireless communication links. In the illustrated embodiment, the medical imaging system 10 includes a first display device 40A that is mounted on the gantry 20, as shown in FIG. 2, and a second display device 40B that is separate (e.g., remote) from the gantry 20. The second display device 40B may be located in a different room than the gantry 20. For example, the second display device 40B may be located in an office of the operator that is separate from the imaging room that houses the medical imaging apparatus 12. The operator may be able to monitor a body contour of the patient during a scan via the second display device 40B. The control circuit 32 may be conductively connected to the first display device 40A via a wire or cable. The control circuit 32 may be connected to the second display device 40B via a network 42. The network 42 may be a wireless network, such that the control circuit 32 generates wireless signals that are transmitted or broadcast to the second display device 40B. Alternatively, the network 42 may be a wired network, such as an Ethernet or Local Area Network (LAN) that connects the control circuit 32 to the second display device 40B.

Alternatively, the control circuit 32 and the memory 38 may be discrete and separate from the medical imaging apparatus 12 (e.g., gantry 20). For example, the control circuit 32 and the memory 38 may be components of a remote computing device, such as a handheld tablet, smartphone, or workstation of the operator (e.g., medical technician, nurse, doctor, or the like). The remote computing device may communicate with one or more components of the medical imaging apparatus 12 via wired cables and/or wireless links.

The medical imaging system 10 may also include a detector motion controller 30 that controls the movement of the detector arms 104 relative to the gantry 20, and also controls the operation of the detection heads within the detector arms 104. The detector motion controller 30 includes one or more processors that operate according to programmed instructions stored on a memory device. For example, the detector motion controller 30 may individually control the radial extension of each of the detector arms 104 between the retracted and extended positions. The detector motion controller 30 may also rotate or orbit the detector arms 104 around the subject 18 as a collective unit. In an embodiment, the detector motion controller 30 may control the detection heads to rotate, pivot, or swivel about respective axes that are substantially parallel to the longitudinal (or depth) axis of the bore 22, and this swiveling allows each of the detection heads to scan the subject 18 with a fan-shaped field of view. The swiveling of the detection heads may be relative to the detector arms 104, such that the detector arms 104 may be stationary while the detection heads swivel within the detector arms 104.

The detector motion controller 30 may be communicatively connected to the control circuit 32. For example, as described herein, the control circuit 32 may generate a control signal that provides the detector motion controller 30 with designated scan positions of the detector arms 104 for an upcoming medical imaging scan of the subject 18. In response to receiving the control signal, the detector motion controller 30 may extend the detector arms 104 relative to the gantry 20 to position the detector arms 104 in the designated scan positions. In another example, the detector motion controller 30 may periodically, or on command, generate a status signal for the control circuit 32 that identifies the current positions of each of the detector arms 104, such as the current extension positions, relative to the gantry 20. Upon receiving the status signal, the control circuit 32 may display graphical detector arms in a gantry visualization on one or more of the display devices 40A, 40B such that the graphical detector arms are displayed in equivalent positions relative to the gantry visualization as the actual detector arms 104 are currently positioned relative to the actual gantry 20. In one example, the gantry visualization along with arm positions may be displayed on one or more of the display devices 40A, 40B along with body contour information such that relative positioning of the detector arms around the patient may be directly visualized and if needed adjusted by the operator.

The medical imaging system 10 also includes an image reconstruction module 34 that is configured to generate medical images from image data generated by the detector arms 104 (and detection heads thereof). The image data from the detector arms 104 may include projection data, positioning data, detected energy data, and/or the like. The image reconstruction module 34 may include one or more processors that operate according to programmed instructions to use NM image reconstruction techniques to generate NM images, such as SPECT images, of the subject 18. The image reconstruction module 34 is communicatively connected to the detector arms 104 to receive the image data. The image reconstruction module 34 may receive the data directly from the detector arms 104, indirectly via the control circuit 32, or indirectly from an acquisition console. The image reconstruction module 34 in an embodiment is connected directly to the gantry 20, such as residing within a common hardware device (e.g., housing) or software module as the control circuit 32. In an alternative embodiment, the image reconstruction module 34 may be remote from the gantry 20, such as residing on a remote server (e.g., in the cloud) and connected to the control circuit 32 via the network 42.

The NM images depict internal elements within the subject 18, and may include a target element or region of interest such as the heart or another organ. The NM images may be three-dimensional or two-dimensional. The NM images may be stored in the memory 38 or another storage medium. The control circuit 32 may access the NM images to display the NM images on one or more of the display devices 40A, 40B, and/or to communicate the NM images remotely via the network 42.

The medical imaging system 10 also includes a user input device 39 that enables an operator to intervene and participate in both the set-up and scan of the medical imaging procedure, as described herein. The user input device 39 may include a touchpad, touchscreen, keyboard, computer mouse, trackball, physical buttons and/or dials, and/or the like. The operator can use the user input device 39 to make user input selections, which are communicated as signals to the control circuit 32. The user input device 39 optionally may be integrated into a common hardware device as one of the display devices 40A, 40B. Optionally, each of the display devices 40A, 40B may be incorporated with a different user input device 39, which allows the operator to communicate and intervene in the medical imaging procedure from either of the locations.

In the illustrated embodiment, the medical imaging apparatus 12 of the medical imaging system 10 includes engagement sensors 44 mounted at the distal ends 106 of the detector arms 104. The engagement sensors 44 are configured to detect physical contact between the detector arms 104 and the subject 18 and any objects associated with the subject 18 within the bore 22, such as linens, the platform 24 of the bed 14 (shown in FIG. 1), and the like. The engagement sensors 44 may be (or include) mechanical switches, such as pressure sensors and other force sensors, and alternatively may be or include proximity sensors, capacitive sensors, or the like. Physical engagement between the detector arms 104 and the subject 18 may degrade the image quality of the NM images, so the engagement sensors 44 are used to ensure that the detector arms 104, although proximate to the subject 18, remain spaced apart from the subject 18 during the medical imaging scan to provide high quality NM images.

The medical imaging apparatus 12 also includes contour sensors 46 in the illustrated embodiment. The contour sensors 46 are mounted to the gantry 20 along the perimeter of the bore 22. The contour sensors 46 optionally may be disposed in the gaps between the detector arms 104, as shown in FIG. 4. The contour sensors 46 may be mounted on a ring on the gantry along the perimeter of the bore 22 such that all sensors are equidistant from the center of the bore 22. The contour sensors 46 are utilized during a contour scan of the subject 18 to provide contour image data. The contour image data generated by the contour sensors 46 is processed by the control circuit 32 to generate a subject shape outline of the subject 18. The subject shape outline is utilized during the medical imaging set-up to determine the designated scan positions of the detector arms 104 for the medical imaging scan. The control circuit 32 also displays the subject shape outline on one or more of the display devices 40A, 40B within the gantry visualization to enable the operator to view and comprehend the subject-gantry geometric relationship. The contour sensors 46 may be any of various types of sensors, such as optical imaging sensors, 3 dimensional cameras, capacitate sensors ultrasound sensors, or the like. The contour sensors 46 and the engagement sensors 44 are communicatively connected to the control circuit 32 to provide the respective sensor data to the control circuit 32.

FIG. 5 illustrates a view of the medical imaging apparatus 12 of the medical imaging system 10 during a contour scan according to an embodiment. The contour scan is performed to generated contour image data, which is used to generate the subject shape outline. The contour scan estimates the shape of the subject 18 within the bore 22 and/or outside the bore (on the bed). The contour scan is performed prior to the medical imaging scan that utilizes the detector arms 104 (shown in FIG. 2) to generate medical image data, such as data for constructing SPECT images. The contour scan may be performed periodically throughout the medical imaging procedure, such as during set-up and subject positioning and also during the medical imaging scan, in order to provide a subject shape outline 206 that is updated in real-time.

In the illustrated embodiment, the gantry 20 includes the contour sensors 46 and light emitting sources 70 installed along a perimeter of the bore 22. The contour sensors 46 in the illustrated embodiment may be light detectors, such as photodiodes. The light emitting sources 70 may be light emitting diodes (LEDs) or the like. The contour sensors 46 and the light emitting sources 70 may be installed in at least one ring 512 around the perimeter of the bore 22 such that the contour sensors 46 are evenly spaced around the perimeter, although FIG. 5 may show less than an entirety of the contour sensors 46 and the light emitting sources 70 that represent a single ring. The gantry 20 may have multiple rings of the contour sensors 46 and light emitting sources 70 at different depths along the bore 22.

In this example, the contour sensors 46 and light emitting sources 70 are shown to be in a ring 512 along the gantry 20. However, another ring with another set of contour sensors and light emitting sources may be positioned outside the gantry and around the bed to determine body contour information and bed position with a patient on the bed prior to transitioning the patient inside the gantry. By capturing body contour information prior to entering the gantry and then again during the scan, sagging of bed over time (during scan) due to patient weight and removal of support as the bed enters the gantry may be identified and rectified.

As an example, a first body contour of the patient on the bed may be captured by the contour sensors and light emitting sources outside the gantry and a second body contour of the same patient may be captured by the contour sensors 46 and light emitting sources 70 along the gantry. In each of the first body contour and the second body contour, a lower portion of the body contour image represents the bed. The controller may compare the first and second body contours to determine if the position of the bed has changed between the initial position of the patient (outside gantry with bed on support) and current position of the patient (inside the gantry). If only a single ring with contour sensors 46 and light emitting sources 70 is present along the gantry, the controller may determine bed sagging by comparing a body contour captured at the onset of the scan to a current body contour captured during the scan to determine any change in the position of the bed due to sagging. Further, the controller may compare the position of the bed to another point of reference such as the floor (which is flat) to determine any sagging occurring during the scan. If it is determined that the bed has sagged, the operator may accordingly adjust detection arm sweep plan to compensate for the change of position of the bed and the patient.

Optionally, the light emitting sources 70 may be controlled to emit light at different times according to an ordered sequence. The light emitted from any particular light emitting source 70 only arrives at some of the contour sensors 46 based on the light emission angle and the shape of the subject 18. As shown in FIG. 5, the solid lines indicate that emitted light rays or beams has impinged upon a contour sensor 46, and the dashed lines indicate that emitted light rays have not impinged upon a contour sensor 46 because such light rays were absorbed, reflected, or otherwise obscured by the subject 18. At a particular time, the control circuit 32 (or other control device performing the contour scan) knows which light emitting source 70 emitted a particular light and receives contour image data indicating which contour sensors 46 received (e.g., detected) that particular light. The control circuit 32 can estimate the shape or contour of the subject 18 using the contour image data across multiple time instances.

In an embodiment, the contour scan may include generating a collection of transaxial contour slices 212 (shown in FIG. 9) of the subject 18. For example, each sequence of light pulses from the light emitting sources 70 may result in a set of contour image data generated by the contour sensors 46. Each set of contour image data may be processed to generate a single transaxial contour slice that represents the contour of the subject 18 at one axial position along the length of the subject 18. In an embodiment, the contour scan may generate the collection of transaxial contour slices by moving the subject 18 at different axial positions within the bore 22. For example, the bed 14 (shown in FIG. 1) may be controlled to move the platform 24 in the axial direction during the contour scan (e.g., while the light emitting sources 70 emit light pulses and the contour sensors 46 detect the light) to generate a multitude of transaxial contour slices of the subject 18 at different axial positions along the length of the subject 18.

In an alternative embodiment, the contour sensors 46 may be range finders installed along the perimeter of the bore 22 instead of using the light emitting sources 70 and the light detectors. For example, the contour sensors 46 may be ultrasonic transducers that emit short pulses of sound and interpret the timing of sound wave echoes to determine the distance to the subject 18. In another example, the contour sensors 46 may be optical transducers, such as laser rangefinders that emit short pulses of light and interpret the timing of reflected light to determine the distance to the subject 18. In yet another alternative embodiment, instead of having the contour sensors 46, the detector arms 104 may generate the contour image data without additional hardware based on detecting scatter radiation during preliminary imaging of the subject 18. The scatter radiation is at energy levels below the original energy peak of the radioactive isotope administered into the subject 18. In another alternative embodiment, the medical imaging apparatus 12 may include a 3D optical camera that generates the contour image data using, for example, infrared light.

FIG. 6 shows an example 600 of a gantry 20 including multiple rings of contour sensors. Light emitting sources 70 and contour sensors 46 are shown in pairs. A first ring 610 of light emitting sources 70 and contour sensors 46 may be positioned along one edge of the gantry 20 proximal to the bed position prior to the scan. A second ring 612 of light emitting sources 70 and contour sensors 46 may be positioned along another, opposite edge of the gantry 20 distal from the subject prior to the scan. The rings 610 and 612 may be placed fully around (within) the gantry bore. The pairs can be axially on either side of any installed detector columns. Each of the first ring 610 and the second ring 612 may be the ring 512 in FIG. 5. In an alternate embodiment, the first ring 610 may be external to the gantry while the second ring 612 may be within the gantry.

As the subject enters the bore, the body contour of a section of the subject may be first captured by the contour sensors 46 included in the first ring 610. As the scan progresses, the subject on the bed may move through the bore of the gantry and the body contour of the same section of the subject may be captured by the contour sensors 46 included in the second ring 612. A dual-ring system as shown in FIG. 6 may also speed up the subject shape estimation process.

FIG. 7A is a pictorial cross-sectional view 700 of an exemplary imaging system including two rings of contour sensors. The subject 18 may be positioned on a bed 14 during the scan. The bed 14 includes a platform that supports the subject 18 (such as a patient to be scanned), which is a human patient in the illustrated embodiment but is not limited to a human patient. For example, the subject 18 may be another living animal or an inanimate object in an alternative embodiment. The bed 14 is able to move longitudinally into the bore 22 for locating the subject 18 in a designated imaging position. The bed 14 includes a lift mechanism 16 that vertically raises and lowers a platform 24 for vertically positioning the subject 18 relative to the gantry 20. The lift mechanism 16 also provides a support for the platform 14 when the subject is outside the gantry 20.

A first ring 610 along a first perimeter of the bore 22 of the gantry 20 may include a first set of light emitting sources and contour sensors. A second ring 612 along a second perimeter of the bore 22 of the gantry 20 may include a second set of light emitting sources and contour sensors. Each of the first ring 610 and the second ring 612 may be positioned between the upper portion 714 and the lower portion 716 of the gantry 20. In this example cross-sectional view, a portion of the subject's anatomy is shown to be scanned via two detectors 724 and 726. The scanning may be simultaneously carried out via all detectors of the imaging system.

The bed 14 may function as a cantilever as it is being supported only on one end (by the lift mechanism 16), the supported end being distal from the gantry 20. When the bed 14 moves through the bore 22, due to gravity and presence of other mechanical constraints, the bed 14 may bend or tilt resulting in sagging. Due to sagging of the bed, the relative position between the subject and detectors may change which may affect absorption of the radiation and the image quality.

In a body contour image reconstructed based on outputs from the contour sensors located along the rings 610 and 612, the contour of the bed is captured in addition to the subject. A first body contour image may be captured by the first ring 610 as the subject passes through the first ring 610 entering the bore 22. After the scan, as the subject 19 exists the bore 22, a second body contour of the subject may be captured by the second ring 612 as the subject passes through the second ring 612. An image analysis of the first body contour and the second body contour may be carried out to determine a relative change in position of the bed (caused by sagging). The difference in position may be determined based on a difference between the first image and the second image and/or based on change in bed position relative to ground. This estimated change in bed position may be used by an image reconstruction module to adjust (correct) the captured images for a single or multiple bed positions.

FIG. 7B shows an example 750 of sagging of the bed 14 as estimated based on inputs from the contour sensors coupled to the first ring 610 and the second ring 612. DS represents the sagging in the bed 14 caused by tilting. This estimation of sagging DS may be directly used during image processing by an image reconstruction module (such as image reconstruction module 34 in FIG. 4) to adjust and generate medical images from image data generated by the detector arms.

In this way, the components of FIGS. 1-7A,B enable a gantry defining a bore configured to receive a subject therein, the gantry including one or more detector arms circumferentially spaced apart along a perimeter of the bore and radially movable relative to the gantry towards and away from the subject, a first set of contour sensors arranged along a first ring mounted on the perimeter proximal to a bed housing the subject prior to a scan, the first set of contour sensors generating a first body contour image of the subject at an onset of the scan, a second set of contour sensors arranged along a second ring mounted on the perimeter of the bore of the gantry proximal to the bed housing the subject prior to a scan, the second set of contour sensors generating a second body contour image of the subject during the scan, and a control circuit including one or more processors to: estimate a change in a position of the bed during the scan based on a comparison of the first body contour image with the second body contour image and adjust one or more medical images of the subject generated from data collected during the scan based on the change in the position of the bed during the scan.

FIG. 8 shows an illustration 800 of the second display device 40B of the medical imaging system 10 and an enlarged inset view 201 showing a portion of a display screen 202 of the second display device 40B displaying a gantry visualization 204 according to an embodiment. The display device 40B in the illustrated embodiment includes or represents a monitor that may be connected to a desktop computer or a laptop computer.

In a non-limiting example, the display device 40B may be located in a separate room than the gantry 20 (shown in FIG. 2), such as in an operator office near an imaging room that houses the gantry 20. In another embodiment, the display device 40B may be a handheld computing device, such as a tablet computer, a smartphone, or the like. The display screen 202 is configured to be viewable to an operator.

The control circuit 32 (shown in FIG. 4) of the medical imaging system 10 (FIG. 4) is configured to generate the gantry visualization 204 that is displayed on the display screen 202. The gantry visualization 204 is a graphical representation of a portion of the medical imaging apparatus 12 (shown in FIG. 2) including the gantry 20 and the bore 22. The gantry visualization 204 is designed to resemble the medical imaging apparatus 12, such that the operator can visualize and understand up-to-date parameters, positioning, and/or operation of the medical imaging apparatus 12 by viewing the display device 40B without viewing the actual medical imaging apparatus 12.

Optionally, the gantry visualization 204 may be part of a graphical user interface, which enables an operator using the user input device 39 (shown in FIG. 4) to interact with and modify the gantry visualization 204 by selecting various items displayed on the gantry visualization 204. Alternatively, the gantry visualization 204 may be non-interactive, and the operator may interact with a separate user interface discrete from the gantry visualization 204 to modify the appearance of the gantry visualization 204.

The gantry visualization 204 shows an end view of the gantry 20 oriented along the longitudinal (or depth) axis of the bore 22. The end view may be a cross-sectional view that shows some components in cross-section. In addition to showing graphical representations of the gantry 20 and the bore 22, the gantry visualization 204 also shows graphical detector arms 208 that graphically represent the detector arms 104.

The gantry visualization 204 also includes a body contour (subject shape outline) 206 of the subject that is disposed within the bore 22 of the gantry 20 for the medical imaging scan. Optionally, the gantry visualization 204 also shows a graphical representation of the platform 24 supporting the subject, as well as a target region indicator 210 that represents a target region of interest of the subject. The target region of interest may be an area of the subject to which the detector arms 104 are focused during medical imaging scan. The target region of interest may include an organ, such as the heart.

The graphical detector arms 208 correspond to the detector arms 104, such that there are twelve graphical detector arms 208 to match the twelve detector arms 104 shown in FIGS. 2 and 3. The graphical detector arms 208 are displayed on the gantry visualization 204 at equivalent or analogous locations along the perimeter of the bore 22 as the corresponding actual (e.g., physical) detector arms 104. The graphical detector arms 208 are radially elongated to extend at least partially into the virtual bore 22. The subject shape outline 206 is displayed on the gantry visualization 204 within the bore 22. The target region indicator 210 is displayed within the body contour 206. The locations of the displayed components relative to the gantry 20 in the gantry visualization 204 are based on the known, measured, estimated, and/or computed locations of the respective components relative to the actual gantry 20. Therefore, the operator can view the gantry visualization 204 to perceive the subject-gantry geometric relationship, such as by viewing the relative positioning of the graphical detector arms 208 to the body contour 206.

One technical effect of the displayed gantry visualization 204 is that the operator can view the subject-gantry geometric relationship without being in a line-of-sight of the bore 22. For example, the operator may even be located in a separate room as the gantry 20 while viewing the gantry visualization 204, which beneficially reduces the operator's exposure to radiation relative to the operator being within the same room as the gantry 20 and peering into the bore 22 to view the positions of the detector arms 104 relative to the subject. By fully visualizing the body contour 206 of the subject and any other machine accessories around the detector arms 104, the operator may determine adjustments to detector positions based on scan protocol and user preferences. In one example, the different detector radial proximity to patient in different areas of the body may be determines, such as a higher clearance between a detector arm and the patient head (relative to a clearance between detector arm and patient feet) may be maintained during the scan. The operator may no longer need to use a table-side measuring apparatus (such as a ruler) to determine relative positioning of the patient and the apparatus and setting up anatomical markers.

During the course of the scan, if there is a change in the position of the subject (such as patient moving), the change may be visible to the operator. The operator may then appropriately change the positon of the bed and/or detector arms without interrupting the scan work flow. In this way, it is possible for the operator to monitor relative positioning between the subject and the detectors without having to be present in the scan room.

Another technical effect of the displayed gantry visualization 204 is that the operator may not be able to view and interpret the subject-gantry geometric relationship in the physical medical imaging apparatus 12. For example, the end view shown in the gantry visualization 204 may not be perceivable to an operator by peering into the bore 22 of the gantry 20. The twelve circumferentially-arranged detector arms 104 may be difficult, if not impossible, for the operator to view based on the number and arrangement of the detector arms 104, and various other components, such as the bed 14 and a housing of the gantry 20 may obstruct the operator's visual access.

Furthermore, even if it is possible for the operator to view all of the detector arms 104 of the gantry 20 in the orientation shown by the gantry visualization 204, such as by acquiring one or more images of the detector arms 104 using a camera, another technical effect of the medical imaging system 10 described herein is that the gantry visualization 204 provides information that is not attainable merely by sight or imaging alone. For example, as described herein in more detail, the subject shape outline 206 displayed in the gantry visualization 204 is generated by aggregating specific subsets of transaxial contour slices depicting the subject in the bore 22. The subject shape outline 206 therefore may have a different shape than the shape of an outline of the subject as seen by a person or camera looking into the bore 22. Furthermore, the gantry visualization 204 may show the graphical detector arms 208 in prospective positions and current positions, whereas a person or camera looking into the bore 22 would only be able to capture the current positions of the detector arms 104. The gantry visualization 204 described herein may integrate various imaging modalities and technology to provide information to the operator about the medical imaging system 10 that may not have been available to the operator using known medical imaging systems and display technology. The medical imaging system 10 provides automated assistance to the operator for the medical imaging procedure, including during the set-up and scanning stages.

FIG. 9 is a diagram showing a side view 900 of a human patient 270 that represents the subject lying on the platform 24 according to an embodiment. An upper portion of the patient is segmented by a plurality of the transaxial contour slices 212 generated during the contour scan of the patient. In an embodiment, the subject shape outline 206 is generated based on a subset of the transaxial contour slices 212. The subset of the transaxial contour slices 212 may correspond to a longitudinal length or depth of the detector arms 104. For example, if the detector arms 104 extend a depth of 40 cm, the subject shape outline 206 may be generated based on a subset of the transaxial contour slices 212 that span at least 40 cm along the length of the patient 270. Assuming uniform slice thickness and spacing between slices 212, the subset may be represented by a given number of consecutive transaxial contour slices 212 along the length, such as 10, 20, 50, or 100 slices 212. In a non-limiting example, each slice 212 has a thickness of about 0.5 cm, so a subset of 80 consecutive, non-overlapping slices 212 spans an axial length of 40 cm along the patient 270.

In the illustrated embodiment, the medical imaging procedure is performed to generate image data of a target region of interest 272 of the patient 270. The target region of interest 272 may be the heart or another organ in the upper torso of the patient 270. According to an embodiment, the medical imaging system 10 provides the operator with the ability to scroll along the axial length of the patient 270 to view different versions of the subject shape outline 206 based on different subsets of the slices 212.

In one example, a computed tomography (CT) scan may be carried out prior to, after or during (in a hybrid system with NM imaging system) a NM imaging. The body contour profile may be used to estimate a center of mass of the subject to be scanned and a height of the bed during the CT scan may be adjusted based on the estimated center of mass. By centering the subject mass against CT Isocenter, optimal patient positioning may be enabled for minimal x-ray radiation exposure (minimal dose) and optimal image quality. Also, scan parameters (such as voltage, current, duration of exposure) during a CT scan may be adjusted by a controller of the CT device or by an operator based on subject characteristics (such as weight, height, etc.) as determined from the body contour.

A lateral view 1000 of an unified body contour of a patient 1002 is shown in FIG. 10A and a frontal view 1020 of the unified body contour of the patient 1002 is shown in FIG. 10B. The operator may be able to toggle between different views (lateral, frontal, sagittal, transverse, etc.) of the body contour. Also, a 2D cross-sectional view of the body contour may be available to the operator. The unified body contour may be generated by integrating a plurality of the transaxial contour slices. Each transaxial body slice may be estimated based on data collected by contour sensors (as described in relation to FIG. 5) during a contour scan. A contour scan may be carried out prior to and during a diagnostic scan (such as via NM imaging or CT scan).

FIG. 11 shows an example method 1100 for generating, saving, and using a body contour of a patient during an imaging procedure This method may be carried out at the onset or at the indication that a new scan is to be carried out at an imaging device such as a nuclear magnetic (NM) imaging device. Method 1100 and all methods described herein may be performed according to instructions stored in the non-transitory memory in a computing device (such as control circuit 32 of FIG. 4) of the imaging system.

At 1102, the routine includes determining if the current imaging procedure to be carried out is a repeat or follow-up from a previous procedure. A repeat or follow-up procedure includes repeating a scan of one or more anatomies of a patient after an interval. In one example, a patient may return to the same clinic for a rest and stress scans in cardiology after an interval of a days or months. A repeat or a follow-up procedure may be indicated to the controller by the operator via a user input device. In one example, the previous procedure may be carried out in the in the same imaging device or a similar device and the body contour profile and scan parameters may be stored in a network cloud or database (identifying the subject's name).

If it is determined that the current imaging procedure is a repeat procedure for the same patient who has been previously scanned, at 1104, the routine includes determining if a body contour is available from the previous scan(s). The body contour may be available in the memory of the same imaging device or from an external device such as a network cloud or a database. The body contour may include a plurality of plurality of the transaxial contour slices of the patient including the anatomy of the patient to be scanned. Each transaxial slice may be generated from data collected by a contour sensor as elaborated with reference to FIG. 5. The body contour may also include a unified body contour generated from a plurality of the transaxial contour slices of the patient. Upon completion of the previous scan, body contour of the patient including the anatomy to be scanned may have be saved in the memory (such as memory storage device 38 in FIG. 4) connected to the control circuit. The scan protocol (parameters) from the previous scan may also be saved in the memory of the imaging device. The body contour profile and the scan protocol may also be saved at an external source such as a network cloud and/or database communicatively coupled to the imaging device. The body contour profile and the scan protocol may be saved corresponding to a subject's information (such as tagged by a name of the patient) in such a way that the body contour profile and scan parameters may be later looked up based on the subject specific information.

If it is determined that a previously saved body contour of the patient is available, at 1106, the body contour may be retrieved from memory of the imaging device or an external source. Also, the scan protocol from the previous scan may be retrieved from the memory or external source. The controller may look-up the body contour profile and the scan protocol using the information of the subject (such as name) used to tag the body contour profile and the scan protocol. At 1108, the subject (such as the patient to be scanned) may be positioned on the bed of the imaging device and scan parameters may be set up by an operator based on the previous scan. As an example, the distance between the head (or toes) and the target scan region (anatomy to be scanned) remains the same between the two scans. The patient may be set-up by the operator on the bed in the same position as the previous visit. In one example, if the position of the patient (on the bed) deviates from the previous position (as retrieved by the controller) an indication may be provided to the operator such that the subject may be repositioned to match that of the previous position. The retrieved body contour may enable automatic detection of positions of the patient's body parts without having to generate a new scan. The scan parameters (also referred herein as protocol) may include bed positions, detector positions, sweep plan of the detectors (change in detector positions during the scan), associated acquisition, and reconstruction parameters per organ\bed position. As an example, a desired radial proximity of the detector heads relative to the patient body may be determined based on the retrieved body contour and scan parameters. As another example, the scan parameters include radial positions of detectors arms of a multi-ram detector relative to a position of the subject on a bed. In one example, a patient may want a larger distance between the head and a detector compared to the distance between the leg and the detector.

Positioning of specific body organs may be identified by the operator based on the 3D patient contour, such as (but not limited to) head, torso, feet. This enables verification of the patient's actual setup against a prescribed protocol, to ensure they match. In this way, the operator may not need to physically mark patient organs or accurately determine the anatomy of the patient prior to the scan. The operator may also determine overall scan time based on the retrieved scan protocol and estimate a workflow plan for the scan.

If at 1102 it is determined that the procedure is not a repeat or follow-up procedure such as if the patient is being scanned for the first time, body contour and scan protocol from a previous scan may not be available in the memory of the imaging system, and the scan may proceed to step 1110. Also, if at 1104 it is determined that even if there were previous scans, a body contour and/or scan protocol may not be available from the previous scan, the routine may directly proceed to 1110.

At 1110, the scan may be started. If scan protocol is not available from a previous scan, the operator may set up a recommended scan protocol for the patient based on the anatomy to be scanned and patient characteristics (height, weight, position of body parts, etc.). A new body contour of the patient may be generated at the onset of the scan and during the scan. The generating the contour data may include capturing contour image data via one or more contour sensors installed along one or more rings mounted on a gantry of the imaging device along a perimeter of a bore of the gantry.

The contour sensors may be installed along one or more rings mounted on a gantry of the imaging device along a perimeter of a bore of the gantry (as shown in FIGS. 5-6). In one example, a first set of contour sensors may be arranged along a first ring mounted on the perimeter proximal to the bed housing the subject prior to a scan, the first set of contour sensors generating a first body contour image of the subject at an onset of the scan and a second set of contour sensors arranged along a second ring mounted on the perimeter of the bore of the gantry proximal to the bed housing the subject prior to a scan, the second set of contour sensors generating a second body contour image of the subject during the scan.

As the motorized bed is moved to position the patient inside the bore of the gantry for the scan, a first body contour may be generated. As the patient moves within the gantry, body contours may be updated at regular intervals. As mentioned, separate body contours may be generated based on inputs from contour sensors installed in separate rings.

At 1112 a visualization of the body contour may be provided to the operator. The visualization of the body contour profile may be in a form of a shape outline of the subject showing anatomy of the subject. Different views of the body contour may be provided such as lateral, frontal, sagittal, transverse, etc. and the operator may be able to toggle between the different views. One or more display devices may be coupled to the control unit and the visualization may be provided on the display devices. As an example, a display device may be positioned in a room separate from the room housing the imaging device such that during the scan, the operator may monitor the body contour (in that display device) without being exposed to radiations in the room housing the imaging device.

In the visualization of the body contour, a relative positioning of detector arms and other peripheral structures coupled to the imaging apparatus may be shown. As an example, position of each detector arm around the patient may be outlined along with the outline of the body contour of the patient. Further, presence of accessories such as head rest, leg support, arm support may be shown in the visualization in the form of outlines which may be annotated.

In the visualization, a difference between the retrieved body contour (from previous scan) and the newly generated body contour may be depicted. As an example, based on the comparative view of the retrieved body contour and the newly generated body contour, the operator may be able to determine changes to the shape and size of the subject such as the subject gaining weight at a certain part of the body.

At 1114, scan parameters may be adjusted based on newly generated body contour and the difference between the retrieved body contour (from previous scan) and the newly generated body contour. As an example, based on the current body contour and/or the difference, the operator may adjust the radial positioning of the detector arms (such as closer or further away from patient body). Also, the operator may make adjustments to the bed position (such as height) such that the patient is at a desirable position within the bore of the gantry.

At 1116, if needed, a position of the patient on the bed may be adjusted. As an example, based on the body contour monitored by the operator, the operator may observe a change in the patient's position on the bed (such as a change in the positioning of an arm) which may affect detector motion. Also, from the dynamically captured body contour (the body contour profile being refreshed periodically), the operator may monitor respiratory motion of the patient. The operator may then remotely instruct the patient via an audio/visual system to change his position on the bed or the operator may go over to the room housing the imaging system to adjust the position of the patient on the bed.

At 1118, the body contour may be updated in real time during the scan. The visualization available to the operator may be refreshed as a new body contour is generated. In one example, a new body contour may be generated every 30 seconds.

At 1120, sagging of the motorized bed on which the patient is being supported may be monitored over the course of the scan. During the scan, when the bed moves through the bore of the gantry, due to gravity and presence of other mechanical constraints, the bed may bend or tilt resulting in sagging. Due to sagging of the bed, the relative position between the subject and detector arms may change which may affect absorption of the radiation and the image quality. Sagging may be detected. A first body contour image may be captured by contour sensors installed around a first ring as the patient passes through the first ring entering the bore. After the scan, as the subject exists the bore, a second body contour of the subject may be captured by contour sensors installed along a second ring as the patient passes through the second ring. In a body contour image, the lower portion is attributed to the bed while the upper portion is attributed to the patient's body. An image analysis of the first body contour and the second body contour may be carried out to determine a relative change in position of the bed as caused by sagging. The difference in position may be determined based on a difference between the first image and the second image and/or based on change in bed position relative to ground.

At 1122, during the scan, based on changes as seen in the body contour and bed position, the operator may adjust the scan parameters (including detector arm positioning), bed position (such as bed height), and patient position (such as how the patients arms and legs are oriented) during the scan, as needed. By monitoring the real time visualization of the body contour relative to arrangement of detector arms and imaging device accessories, the operator may adjust scan parameters as needed and overall improve the scanning process.

At 1124, upon completion of a scan, one or more body contours generated during the scan may be saved in the memory of the imaging device. During a future scan of the same patient, this body contour may be retrieved to improve workflow.

In this way, during a scan of a patient, a body contour of the patient may be generated based on inputs from one or more body contour sensors housed in a perimeter of a bore of a gantry, a visualization of the generated body contour relative to one or more detector arms of the imaging device may be provided to an operator, the visualization including positions of organs of the patient within the body contour; and based on the generated body contour, the operator may adjust respective positions of the one or more detector arms and set a scan range for each of the one or more detector arms over a course of the scan.

In one example, a method for an imaging device, comprises: prior to a start of an imaging scan, retrieving an initial body contour profile of a subject to be scanned, setting up scan parameters for the imaging scan based on the retrieved body contour profile, generating body contour profile during the imaging scan, providing visualization of the generated body contour profile to an operator. In the preceding example, additionally or optionally, the scan parameters include radial positions of detectors arms of a multi-ram detector relative to a position of the subject on a bed. In any or all of the preceding examples, additionally or optionally, the scan parameters further include a sweep plan for the detector arms for a single bed position or a sequence of bed positions during the imaging scan. In any or all of the preceding examples, additionally or optionally, the visualization of the body contour profile is in a form of a shape outline of the subject showing anatomy of the subject. In any or all of the preceding examples, additionally or optionally, the visualization shows a lateral and/or frontal view of the shape outline of the subject, and a difference between the retrieved body contour profile and the generated body contour profile. In any or all of the preceding examples, additionally or optionally, the visualization shows a relative positioning of each of the subject, the detector arms, and accessories coupled to the imaging device. In any or all of the preceding examples, additionally or optionally, the visualization is available on a device located outside or inside a room housing the imaging device, the operator controlling the device. In any or all of the preceding examples, additionally or optionally, the generating the contour data includes capturing contour image data via one or more contour sensors installed along one or more rings mounted on a gantry of the imaging device along a perimeter of a bore of the gantry. In any or all of the preceding examples, additionally or optionally, the contour sensors including one or more of imaging sensors, 3 dimensional cameras, capacitate sensors, and ultrasound sensors. In any or all of the preceding examples, additionally or optionally, generating body the contour data during the imaging scan includes updating the body contour profile at a threshold interval. In any or all of the preceding examples, additionally or optionally, adjusting scan parameters include changing one or more of the radial positions of detector arms, sweep plan for the detector arms, the single bed position, and the sequence of bed positions in response to a change in a position of the subject during the scan, the change in the position of the subject estimated based on the generated body contour profile during the imaging scan. In any or all of the preceding examples, additionally or optionally, the initial body contour profile is generated during a previous imaging scan of the subject using the imaging device.

Another example system for an imaging device comprises: a gantry defining a bore configured to receive a subject therein, the gantry including one or more detector arms circumferentially spaced apart along a perimeter of the bore and radially movable relative to the gantry towards and away from the subject, a first set of contour sensors arranged along a first ring mounted on the perimeter proximal to a bed housing the subject prior to a scan, the first set of contour sensors generating a first body contour image of the subject at an onset of the scan, a second set of contour sensors arranged along a second ring mounted on the perimeter of the bore of the gantry proximal to the bed housing the subject prior to a scan, the second set of contour sensors generating a second body contour image of the subject during the scan, and a control circuit including one or more processors to: estimate a change in a position of the bed during the scan based on a comparison of the first body contour image with the second body contour image and adjust one or more medical images of the subject generated from data collected during the scan based on the change in the position of the bed during the scan. In the preceding example, additionally or optionally, the first body contour image includes a first position of the bed and a first position of the subject, and wherein the second body contour image includes a second position of the bed and a second position of the subject, the change in the position of the bed estimated based on the first position of the bed and the second position of the bed. In any or all of the preceding examples, additionally or optionally, the bed is a cantilever supported on a side distal from the gantry. In any or all of the preceding examples, additionally or optionally, the imaging system is a nuclear medicine imaging system.

In yet another example, an example method for an imaging device, comprises: during a scan of a patient, generating a body contour of the patient based on inputs from one or more body contour sensors housed in a perimeter of a bore of a gantry, providing visualization of the generated body contour relative to one or more detector arms of the imaging device to an operator, the visualization including positions of organs of the patient within the body contour. In the preceding example, the method further comprising, additionally or optionally, based on the generated body contour, adjusting scan parameters including voltage, current, and duration of x-ray exposure during a computed tomography scan carried out prior to, after, or during the scan of the patient via the imaging device. In any or all of the preceding examples, the method further comprising, additionally or optionally, upon completion of the scan, saving the generated body contour in a memory coupled to the imaging device. In any or all of the preceding examples, additionally or optionally, the generated body contour is updated in real-time at predetermined time intervals.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for an imaging device, comprising:
prior to a start of an imaging scan,
retrieving an initial body contour profile of a subject to be scanned;
setting up scan parameters for the imaging scan based on the retrieved body contour profile;
generating body contour profile during the imaging scan; and
providing visualization of the generated body contour profile to an operator

2. The method of claim 1, wherein the scan parameters include radial positions of detectors arms of a multi-ram detector relative to a position of the subject on a bed.

3. The method of claim 2, wherein the scan parameters further include a sweep plan for the detector arms for a single bed position or a sequence of bed positions during the imaging scan.

4. The method of claim 2, wherein the visualization of the body contour profile is in a form of a shape outline of the subject showing anatomy of the subject.

5. The method of claim 4, wherein the visualization shows a lateral and/or frontal view of the shape outline of the subject, and a difference between the retrieved body contour profile and the generated body contour profile.

6. The method of claim 4, wherein the visualization shows a relative positioning of each of the subject, the detector arms, and accessories coupled to the imaging device.

7. The method of claim 1, wherein the visualization is available on a device located outside or inside a room housing the imaging device, the operator controlling the device.

8. The method of claim 1, wherein the generating the contour data includes capturing contour image data via one or more contour sensors installed along one or more rings mounted on a gantry of the imaging device along a perimeter of a bore of the gantry.

9. The method of claim 8, wherein the contour sensors including one or more of imaging sensors, 3 dimensional cameras, capacitate sensors, and ultrasound sensors.

10. The method of claim 1, wherein generating body the contour data during the imaging scan includes updating the body contour profile at a threshold interval.

11. The method of claim 1, wherein adjusting scan parameters include changing one or more of the radial positions of detector arms, sweep plan for the detector arms, the single bed position, and the sequence of bed positions in response to a change in a position of the subject during the scan, the change in the position of the subject estimated based on the generated body contour profile during the imaging scan.

12. The method of claim 1, wherein the initial body contour profile is generated during a previous imaging scan of the subject using the imaging device.

13. A system for an imaging device, comprising:
a gantry defining a bore configured to receive a subject therein, the gantry including one or more detector arms circumferentially spaced apart along a perimeter of the bore and radially movable relative to the gantry towards and away from the subject;
a first set of contour sensors arranged along a first ring mounted on the perimeter proximal to a bed housing the subject prior to a scan, the first set of contour sensors generating a first body contour image of the subject at an onset of the scan;
a second set of contour sensors arranged along a second ring mounted on the perimeter of the bore of the gantry proximal to the bed housing the subject prior to a scan, the second set of contour sensors generating a second body contour image of the subject during the scan; and
a control circuit including one or more processors to:
estimate a change in a position of the bed during the scan based on a comparison of the first body contour image with the second body contour image and
adjust one or more medical images of the subject generated from data collected during the scan based on the change in the position of the bed during the scan.

14. The system of claim 13, wherein the first body contour image includes a first position of the bed and a first position of the subject, and wherein the second body contour image includes a second position of the bed and a second position of the subject, the change in the position of the bed estimated based on the first position of the bed and the second position of the bed.

15. The system of claim 13, wherein the bed is a cantilever supported on a side distal from the gantry.

16. The system of claim 13, wherein the imaging system is a nuclear medicine imaging system.

17. A method for an imaging device, comprising:
during a scan of a patient,
generating a body contour of the patient based on inputs from one or more body contour sensors housed in a perimeter of a bore of a gantry;
providing visualization of the generated body contour relative to one or more detector arms of the imaging device to an operator, the visualization including positions of organs of the patient within the body contour.

18. The method of claim 17, further comprising, based on the generated body contour, adjusting setting scan parameters including voltage, current, and duration of x-ray exposure during a computed tomography scan carried out prior to, after, or during the scan of the patient via the imaging device.

19. The method of claim 17, further comprising, upon completion of the scan, saving the generated body contour in a memory communicatively coupled to the imaging device.

20. The method of claim 17, wherein the generated body contour is updated in real-time at predetermined time intervals.
